(19) United States
(12) Patent Application Publication
(10) Pub. No.: US 2009/0157138 A1
(43) Pub. Date: Jun. 18, 2009

(54) Methods And Apparatus For Treating Ileus Condition Using Electrical Signals

(75) Inventors: Joseph P. ERRICO, Green Brook, NJ (US); Steven MENDEZ, Chester, NJ (US); Peter S. STAATS, Colts Neck, NJ (US)

Correspondence Address: GIBSON & DERNIER L.L.P., 900 ROUTE 9 NORTH, SUITE 504, WOODBRIDGE, NJ, 07095, US

(73) Assignee: ElectroCore, Inc., Morris Plains, NJ (US)

(21) Appl. No.: 12/246,605

(22) Filed: Oct. 7, 2008

Related U.S. Application Data
(63) Continuation-in-part of application No. 11/735,709, filed on Apr. 16, 2007.
(60) Provisional application No. 60/792,823, filed on Apr. 18, 2006.
(60) Provisional application No. 60/978,240, filed on Oct. 8, 2007.

(51) Int. Cl. A61N 1/36

(52) U.S. Cl. 607/40

(57) ABSTRACT

A method of treating a temporary arrest of intestinal peristalsis includes inducing at least one of an electric current, an electric field and an electromagnetic field in a sympathetic nerve chain of a mammal to block and/or modulate inhibitory nerve signals thereof such that intestinal peristalsis function is at least partially improved.

### CROSS REFERENCE TO RELATED APPLICATIONS

This application is a continuation-in-part of U.S. patent application Ser. No. 11/735,709, filed Apr. 16, 2007, which in turn claims the benefit of U.S. Provisional Patent Application No. 60/792,823, filed Apr. 18, 2006, and this application claims the benefit of U.S. Provisional Patent Application No. 60/978,240, filed Oct. 8, 2007, the entire disclosures of which are hereby incorporated by reference.

### BACKGROUND OF THE INVENTION

The present invention relates to the field of delivery of electrical impulses to bodily tissues for therapeutic purposes, and more specifically to devices and methods for treating conditions associated with a temporary arrest of intestinal peristalsis, such as paralytic Ileus, adynamic Ileus, and/or paresis.

The use of electrical stimulation for treatment of medical conditions has been well known in the art for nearly two thousand years. One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed. Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

There are two types of intestinal obstructions, mechanical and non-mechanical. Mechanical obstructions occur because the bowel is physically blocked and its contents can not pass the point of the obstruction. This happens when the bowel twists on itself (volvulus) or as the result of hernias, impacted feces, abnormal tissue growth, or the presence of foreign bodies in the intestines. Ileus is a partial or complete non-mechanical blockage of the small and/or large intestine. Unlike mechanical obstruction, non-mechanical obstruction, Ileus or paralytic Ileus, occurs because peristalsis stops. Peristalsis is the rhythmic contraction that moves material through the bowel.

Ileus may be associated with an infection of the membrane lining the abdomen, such as intraperitoneal or retroperitoneal infection, which is one of the major causes of bowel obstruction in infants and children. Ileus may be produced by mesenteric ischemia, by arterial or venous injury, by retroperitoneal or intra-abdominal hematomas, after intra-abdominal surgery, in association with renal or thoracic disease, or by metabolic disturbances (e.g., hypokalemia).

Gastric and colonic motility disturbances after abdominal surgery are largely a result of abdominal manipulation. The small bowel is largely unaffected, and motility and absorption are normal within a few hours after operation. Stomach emptying is usually impaired for about twenty four hours, but the colon may remain inert for about forty-eight to seventy-two hours (and in some cases 4-7 days). These findings may be confirmed by daily plain x-rays of the abdomen taken postoperatively; they show gas accumulating in the colon but not in the small bowel. Activity tends to return to the cecum before it returns to the sigmoid. Accumulation of gas in the small bowel implies that a complication (e.g., obstruction, peritonitis) has developed.

Symptoms and signs of Ileus include abdominal distention, vomiting, obstipation, and cramps. Auscultation usually reveals a silent abdomen or minimal peristalsis. X-rays may show gaseous distention of isolated segments of both small and large bowel. At times, the major distention may be in the colon. When a doctor listens with a stethoscope to the abdomen there will be few or no bowel sounds, indicating that the intestine has stopped functioning. Ileus can be confirmed by x rays of the abdomen, computed tomography scans (CT scans), or ultrasound. It may be necessary to do more invasive tests, such as a barium enema or upper GI series, if the obstruction is mechanical. Blood tests also are useful in diagnosing paralytic Ileus.

Conventionally, patients may be treated with supervised bed rest in a hospital, and bowel rest—where nothing is taken by mouth and patients are fed intravenously or through the use of a nasogastric tube. In some cases, continuous nasogastric suction may be employed, in which a tube inserted through the nose, down the throat, and into the stomach A similar tube can be inserted in the intestine. The contents are then suctioned out. In some cases, especially where there is a mechanical obstruction, surgery may be necessary.

Intravenous fluids and electrolytes may be administered, and a minimal amount of sedatives. An adequate serum K level (>4 mEq/L [>4 mmol/L]) is usually important. Sometimes colonic Ileus can be relieved by colonoscopic decompression. Cecostomy is rarely required.

Drug therapies that promote intestinal motility (ability of the intestine to move spontaneously), such as cisapride and vasopressin (Pitressin), are sometimes prescribed. Some reported opiate therapies (such as alvimopan) are directed to inhibiting sympathetic nerve transmission to improve intestinal peristalsis.

Alternative practitioners offer few treatment suggestions, but focus on prevention by keeping the bowels healthy through eating a good diet, high in fiber and low in fat If the case is not a medical emergency, homeopathic treatment and traditional Chinese medicine can recommend therapies that may help to reinstate peristalsis.

Ileus persisting for more than about one week usually involves a mechanical obstructive cause, and laparotomy is usually considered. Colonoscopic decompression may be helpful in cases of pseudo-obstruction (Ogilvie's syndrome), which consists of apparent obstruction at the splenic flexure, although no associated cause is found by barium enema or colonoscopy for the failure of gas and feces to pass.

Unfortunately, many lengthy post operative stays in the hospital are associated with Ileus, where the patient simply cannot be discharged until his bowels move. The clinical consequences of postoperative Ileus can be profound. Patients with Ileus are immobilized, have discomfort and pain, and are at increased risk for pulmonary complications. Ileus also enhances catabolism because of poor nutrition. It has been reported in the 1990's that, overall, Ileus prolongs hospital stays, costing $750 million annually in the United States. Thus, it stands to reason that the healthcare costs associated with Ileus over a decade later are much higher. The relatively high medical costs associated with such post operative hospital stays are clearly undesirable, not to mention patient discomfort, and other complications. There are not, however, any commercially available medical equipment that can treat Ileus. It is therefore desirable to avoid the complications associated with the temporary arrest of intestinal peristalsis, particularly that resulting from abdominal surgery, and provide equipment capable of delivering an internal or external treatment to reduce and/or eliminate the pathological responses that are associated with Ileus.

### SUMMARY OF THE INVENTION

Post-operative ileus (POI) is a common transient bowel dysmotility. POI is a frequent complication seen in a preponderance of major abdominal surgeries, as well as one of the most frequently encountered sequela of intra-peritoneal chemotherapy. The signs and symptoms associated with POI include abdominal pain and distension, reduced borborygmi, vomiting, nausea, early satiety, and an increased transit time for the passage of flatus and/or stool. POI frequently results in prolonged hospital stays as a consequence of gastrointestinal (GI) complications. Recent estimates of the medical costs incurred due to these complications exceed $1 billion annually. Clinical complications associated with POI include an increase in nasogastric tube reinsertion, intravenous volume maintenance and/or hydration, added nursing care, additional laboratory testing, increased re-admission, and more days in-hospital.

The use of spinal cord stimulation (SCS) in the management of pain syndromes is a minimally invasive and reversible, implantable neurostimulation modality. This modality has been shown clinically to be effective over a range of maladies including ischemic heart disease—refractory angina pectoris, low back pain with radiculopathy, failed-back surgery syndrome (FBSS), abdominal pain, peripheral vascular disease, and complex regional pain syndrome (CRPS). Reports of SCS clinical success range from 50% to 80% with reductions in medication requirements as well as improvements in pain intensity scores, quality of life (QOL) enhancements, corrected function, and bolstered chances of returning to work.

Recent reviews in the art have discussed the potential application of electrical stimulation of the end organ, namely the stomach, small intestine or colon to improve motility. SCs may also be a useful treatment modality for dysmotility, particularly delayed gastric and intestinal motility following surgery. SCS may accelerate motility in patients with POI, following therapeutic SCS for chronic pain, patients report increased bowel movements and relief from severe constipation. Colonic motility have been assessed by others in two patients that underwent SCS for neurogenic bladder with concomitant severe constipation. Both received SCS at the level of the 8^{th} and 9^{th} thoracic vertebrae and reported spontaneous defecation within 12 hours and increased weekly bowel movements. In another case report, two patients received permanent spinal cord stimulator implants, where the generators were placed for pain management. While the adverse GI symptomatology varied between each patient, common to both was persistent diarrhea associated with stimulator use. These GI side effects were severe enough that both patients had the permanent stimulators removed in spite of excellent pain coverage. In contrast, a single case report found that SCS was able to eliminate diarrhea in a patient with irritable bowel syndrome (IBS), even after the beneficial effect on pain management abated. Taken together, these clinical reports in the art support a previously unconsidered association between SCS and alterations in gastrointestinal motility.

In accordance with one or more embodiments of the present invention, methods and apparatus for treating the temporary arrest of intestinal peristalsis provide for: inducing at least one of an electric current, an electric field and an electromagnetic field in a sympathetic nerve chain of a mammal to modulate and/or block inhibitory nerve signals thereof such that intestinal peristalsis function is at least partially improved.

The electric current, electric field and/or electromagnetic field may be applied to at least one of the celiac ganglia, cervical ganglia, and thoracic ganglia of the sympathetic nerve chain. Alternatively or additionally, the electric current, electric field and/or electromagnetic field may be applied to at least a portion of the splanchnic nerves of the sympathetic nerve chain, and/or the spinal levels from T5 to L2.

The methods and apparatus may further provide for inducing the current and/or field(s) by applying at least one electrical impulse to one or more emitters. The one or more emitters may be disposed at least one of subcutaneously and percutaneously to direct the field(s) to the spinal cord and/or sympathetic nerve chain. The one or more emitters may include at least one of contact electrodes, capacitive coupling electrodes, and inductive coils. Drive signals may be applied to the one or more emitters to produce the at least one impulse and induce the current and/or field(s). The drive signals may include at least one of sine waves, square waves, triangle waves, exponential waves, and complex impulses.

The drive signals inducing the current and/or fields preferably have a frequency, an amplitude, a duty cycle, a pulse width, a pulse shape, etc. selected to influence the therapeutic result, namely modulating some or all of the nerve transmissions in the sympathetic nerve chain. By way of example, the parameters of the drive signal may include a square wave profile having a frequency of about 10 Hz or greater, such as between about 15 Hz to 200 Hz, or between about 25 Hz to about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIGS. 1-2 are schematic diagrams of the human autonomic nervous system, illustrating sympathetic fibers, spinal nerve root fibers, and cranial nerves;

FIG. 3 is another schematic diagram of the human autonomic nervous system including an apparatus for electrically stimulating, blocking and/or modulating the sympathetic fibers and/or spinal nerve fibers;

FIG. 4 is a graphical illustration of an electrical signal profile that may be used to treat disorders through neuromuscular modulation in accordance with one or more embodiments of the present invention;

FIG. 5 is a graphical illustration of the results of gastric emptying by treating the spinal cords of laboratory rats with electrical signal profiles in accordance with one or more embodiments of the present invention;

FIG. 6 is a graphical illustration of the results of small intestinal transmit (geometric center) by treating the spinal cords of laboratory rats with an electrical signal profiles in accordance with one or more embodiments of the present invention;

FIG. 7 is a graphical illustration of the results of small intestinal transmit (head of meal) by treating the spinal cords of laboratory rats with an electrical signal profiles in accordance with one or more embodiments of the present invention;

FIG. 8 is a graphical illustration of the results of gastric emptying in further experiments by treating the spinal cords of laboratory rats with electrical signal profiles in accordance with one or more embodiments of the present invention;

FIG. 9 is a graphical illustration of the results of small intestinal transmit (head of meal) in the further experiments by treating the spinal cords of laboratory rats with an electrical signal profiles in accordance with one or more embodiments of the present invention; and

FIG. 10 is a graphical illustration of the results of small intestinal transmit (geometric center) of the further experiments by treating the spinal cords of laboratory rats with an electrical signal profiles in accordance with one or more embodiments of the present invention.

### DETAILED DESCRIPTION OF THE PREFERRED EMBODIMENTS

Ileus occurs from hypomotility of the gastrointestinal tract in the absence of a mechanical bowel obstruction. This suggests that the muscle of the bowel wall is transiently impaired and fails to transport intestinal contents. This lack of coordinated propulsive action leads to the accumulation of both gas and fluids within the bowel. Although Ileus has numerous causes, the postoperative state is the most common scenario for Ileus development. Frequently, Ileus occurs after intraperitoneal operations, but it may also occur after retroperitoneal and extra-abdominal surgery. The longest duration of Ileus has been reported to occur after colonic surgery.

According to some hypotheses, postoperative Ileus is mediated via activation of inhibitory spinal reflex arcs. Anatomically, three distinct reflexes are involved: ultrashort reflexes confined to the bowel wall, short reflexes involving prevertebral ganglia, and long reflexes involving the spinal cord. Spinal anesthesia, abdominal sympathectomy, and nerve-cutting techniques have been demonstrated to either prevent or attenuate the development of Ileus. The surgical stress response leads to systemic generation of endocrine and inflammatory mediators that also promote the development of Ileus. Rat models have shown that laparotomy, eventration, and bowel compression lead to increased numbers of macrophages, monocytes, dendritic cells, T cells, natural killer cells, and mast cells, as demonstrated by immunohistochemistry. Calcitonin gene-related peptide, nitric oxide, vasoactive intestinal peptide, and substance p function as inhibitory neurotransmitters in the bowel nervous system. Nitric oxide and vasoactive intestinal peptide inhibitors and substance p receptor antagonists have been demonstrated to improve gastrointestinal function.

In accordance with one or more embodiments of the present invention, a method of treating a temporary arrest of intestinal peristalsis (such as Ileus) includes inducing an electric current, an electric field and/or an electromagnetic field in a sympathetic nerve chain of a mammal to block inhibitory nerve signals thereof such that intestinal peristalsis function is at least partially improved. The electric current, electric field and/or electromagnetic field may be induced by way of externally disposed apparatus, such as a control unit (including a drive signal generator) and percutaneous and/or subcutaneous emitters, such as contact electrodes, capacitive coupling electrodes and/or inductive coils. (Alternative embodiments of the present invention may provide for entirely subcutaneous components, including the control unit, signal generator, and/or the electrodes/coils).

The emitters (whether disposed percutaneously or subcutaneously) are preferably located to direct the current, electric and/or electromagnetic fields to or toward one or more portions of the spinal cord and/or sympathetic nerve chain. Particular locations for the emitters include one or more areas such that the electric current, electric field and/or electromagnetic field is applied to at least one of the celiac ganglia, cervical ganglia, and thoracic ganglia of the sympathetic nerve chain. Alternative and/or additional locations for the emitters include one or more areas such that the electric current, electric field and/or electromagnetic field is applied to at least a portion of the splanchnic nerves of the sympathetic nerve chain, and/or one or more of the spinal levels from T5 to L2.

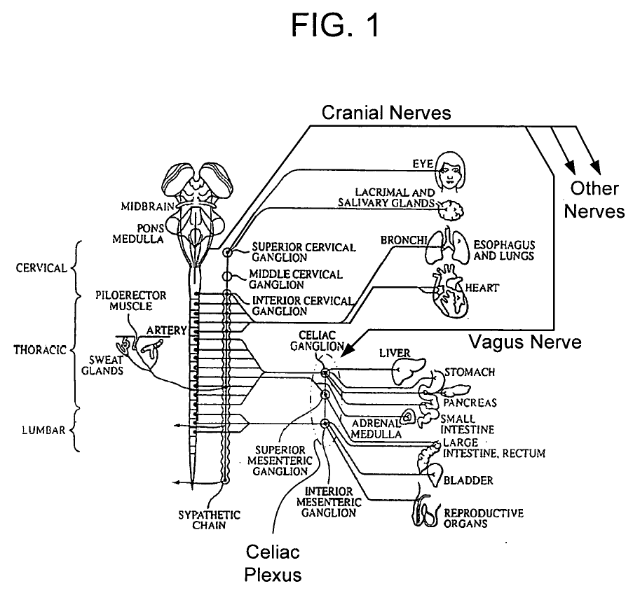

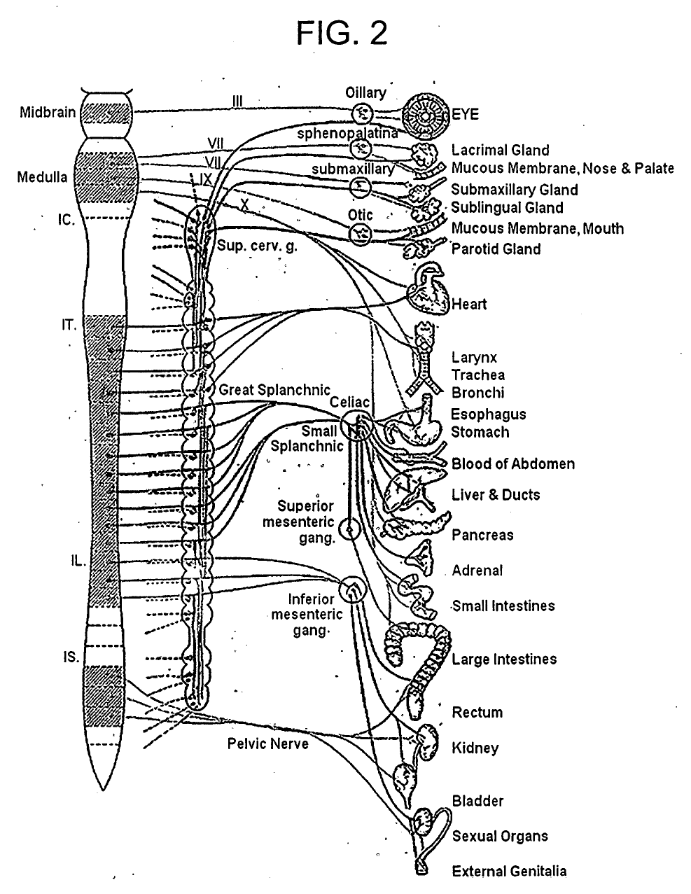

In connection with the location(s) of the emitter(s), a discussion of the human autonomic nervous system, including sympathetic fibers and parasympathetic fibers will now be provided with reference to FIGS. 1-3. The sympathetic nerve fibers, along with many of the spinal cord's nerve root fibers, and the cranial nerves that innervate tissue in the thoracic and abdominal cavities are sometimes referred to as the autonomic, or vegetative, nervous system. The sympathetic, spinal, and cranial nerves all have couplings to the central nervous system, generally in the primitive regions of the brain, however, these components have direct effects over many regions of the brain, including the frontal cortex, thalamus, hypothalamus, hippocampus, and cerebellum. The central components of the spinal cord and the sympathetic nerve chain extend into the periphery of the autonomic nervous system from their cranial base to the coccyx, essentially passing down the entire spinal column, including the cervical, thoracic and lumbar regions. The sympathetic chain extends on the anterior of the column, while the spinal cord components pass through the spinal canal. The cranial nerves, the one most innervating of the rest of the body being the vagus nerve, passes through the dura mater into the neck, and then along the carotid and into the thoracic and abdominal cavities, generally following structures like the esophagus, the aorta, and the stomach wall.

Because the autonomic nervous system has both afferent and efferent components, modulation of its fibers can affect both the end organs (efferent) as well as the brain structure to which the afferents fibers are ultimately coupled within the brain.

Although sympathetic and cranial fibers (axons) transmit impulses producing a wide variety of differing effects, their component neurons are morphologically similar. They are smallish, ovoid, multipolar cells with myelinated axons and a variable number of dendrites. All the fibers form synapses in peripheral ganglia, and the unmyelinated axons of the ganglionic neurons convey impulses to the viscera, vessels and other structures innervated. Because of this arrangement, the axons of the autonomic nerve cells in the nuclei of the cranial nerves, in the thoracolumbar lateral comual cells, and in the gray matter of the sacral spinal segments are termed preganglionic sympathetic nerve fibers, while those of the ganglion cells are termed postganglionic sympathetic nerve fibers. These postganglionic sympathetic nerve fibers converge, in small nodes of nerve cells, called ganglia that lie alongside the vertebral bodies in the neck, chest, and abdomen. The effects of the ganglia as part of the autonomic system are extensive. Their effects range from the control of insulin production, cholesterol production, bile production, satiety, other digestive functions, blood pressure, vascular tone, heart rate, sweat, body heat, blood glucose levels, and sexual arousal.

The parasympathetic group lies predominately in the cranial and cervical region, while the sympathetic group lies predominantly in the lower cervical, and thoracolumbar and sacral regions. The sympathetic peripheral nervous system is comprised of the sympathetic ganglia that are ovoid/bulb like structures (bulbs) and the paravertebral sympathetic chain (cord that connects the bulbs). The sympathetic ganglia include the central ganglia and the collateral ganglia.

The central ganglia are located in the cervical portion, the thoracic portion, the lumbar portion, and the sacral portion. The cervical portion of the sympathetic system includes the superior cervical ganglion, the middle cervical ganglion, and the interior cervical ganglion.

The thoracic portion of the sympathetic system includes twelve ganglia, five upper ganglia and seven lower ganglia. The seven lower ganglia distribute filaments to the aorta, and unite to form the greater, the lesser, and the lowest splanchnic nerves. The greater splanchnic nerve (splanchnicus major) is formed by branches from the fifth to the ninth or tenth thoracic ganglia, but the fibers in the higher roots may be traced upward in the sympathetic trunk as far as the first or second thoracic ganglion. The greater splanchnic nerve descends on the bodies of the vertebrae, perforates the crus of the diaphragm, and ends in the celiac ganglion of the celiac plexus. The lesser splanchnic nerve (splanchnicus minor) is formed by filaments from the ninth and tenth, and sometimes the eleventh thoracic ganglia, and from the cord between them. The lesser splanchnic nerve pierces the diaphragm with the preceding nerve, and joins the aorticorenal ganglion. The lowest splanchnic nerve (splanchnicus imus) arises from the last thoracic ganglion, and, piercing the diaphragm, ends in the renal plexus.

The lumbar portion of the sympathetic system usually includes four lumbar ganglia, connected together by interganglionic cords. The lumbar portion is continuous above, with the thoracic portion beneath the medial lumbocostal arch, and below with the pelvic portion behind the common iliac artery. Gray rami communicantes pass from all the ganglia to the lumbar spinal nerves. The first and second, and sometimes the third, lumbar nerves send white rami communicantes to the corresponding ganglia.

The sacral portion of the sympathetic system is situated in front of the sacrum, medial to the anterior sacral foramina. The sacral portion includes four or five small sacral ganglia, connected together by interganglionic cords, and continuous above with the abdominal portion. Below, the two pelvic sympathetic trunks converge, and end on the front of the coccyx in a small ganglion.

The collateral ganglia include the three great gangliated plexuses, called, the cardiac, the celiac (solar or epigastric), and the hypogastric plexuses. The great plexuses are respectively situated in front of the vertebral column in the thoracic, abdominal, and pelvic regions. They consist of collections of nerves and ganglia; the nerves being derived from the sympathetic trunks and from the cerebrospinal nerves. They distribute branches to the viscera.

The celiac plexus is the largest of the three great sympathetic plexuses and is located at the upper part of the first lumbar vertebra. The celiac plexus is composed of the celiac ganglia and a network of nerve fibers uniting them together. The celiac plexus and the ganglia receive the greater and lesser splanchnic nerves of both sides and some filaments from the right vagus nerve. The celiac plexus gives off numerous secondary plexuses along the neighboring arteries. The upper part of each celiac ganglion is joined by the greater splanchnic nerve, while the lower part, which is segmented off and named the aorticorenal ganglion, receives the lesser splanchnic nerve and gives off the greater part of the renal plexus.

The secondary plexuses associated with the celiac plexus consist of the phrenic, hepatic, lineal, superior gastric, suprarenal, renal, spermatic, superior mesenteric, abdominal aortic, and inferior mesenteric. The phrenic plexus emanates from the upper part of the celiac ganglion and accompanies the inferior phrenic artery to the diaphragm, with some filaments passing to the suprarenal gland and branches going to the inferior vena cava, and the suprarenal and hepatic plexuses. The hepatic plexus emanates from the celiac plexus and receives filaments from the left vagus and right phrenic nerves. The hepatic plexus accompanies the hepatic artery and ramifies upon its branches those of the portal vein in the substance of the liver. Branches from hepatic plexus accompany the hepatic artery, the gastroduodenal artery, and the right gastroepiploic artery along the greater curvature of the stomach.

The lienal plexus is formed from the celiac plexus, the left celiac ganglion, and from the right vagus nerve. The lienal plexus accompanies the lienal artery to the spleen, giving off subsidiary plexuses along the various branches of the artery. The superior gastric plexus accompanies the left gastric artery along the lesser curvature of the stomach, and joins with branches from the left vagus nerve. The suprarenal plexus is formed from the celiac plexus, from the celiac ganglion, and from the phrenic and greater splanchnic nerves. The suprarenal plexus supplies the suprarenal gland. The renal plexus is formed from the celiac plexus, the aorticorenal ganglion, and the aortic plexus, and is joined by the smallest splanchnic nerve. The nerves from the suprarenal plexus accompany the branches of the renal artery into the kidney, the spermatic plexus, and the inferior vena cava. The spermatic plexus is formed from the renal plexus and aortic plexus. The spermatic plexus accompanies the internal spermatic artery to the testis (in the male) and the ovarian plexus, the ovary, and the uterus (in the female). The superior mesenteric plexus is formed from the lower part of the celiac plexus and receives branches from the right vagus nerve.

The superior mesenteric plexus surrounds the superior mesenteric artery and accompanies it into the mesentery, the pancreas, the small intestine, and the great intestine. The abdominal aortic plexus is formed from the celiac plexus and ganglia, and the lumbar ganglia. The abdominal aortic plexus is situated upon the sides and front of the aorta, between the origins of the superior and inferior mesenteric arteries, and distributes filaments to the inferior vena cava. The inferior mesenteric plexus is formed from the aortic plexus. The inferior mesenteric plexus surrounds the inferior mesenteric artery, the descending and sigmoid parts of the colon and the rectum.

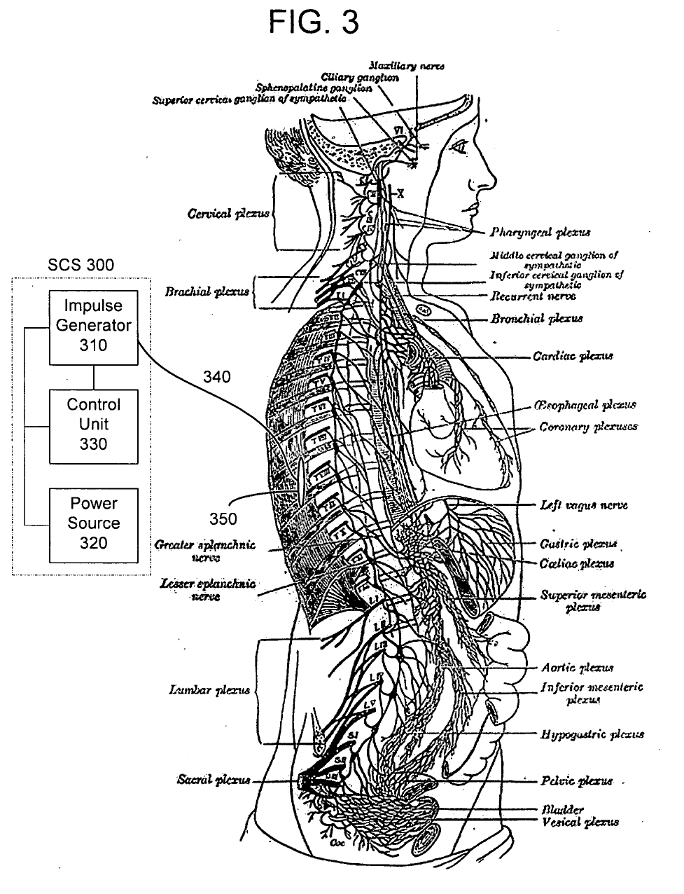

The current and/or fields may be induced by applying at least one electrical impulse to the emitters, such as by using the signal generator to apply the drive signals to the emitters. Particular reference is now made to FIG. 3, which illustrates a view of the anatomy and a spinal cord nerve stimulation device (SCS) 300 for blocking and/or modulating inhibitory nerve signals such that intestinal peristalsis function is at least partially improved. The SCS device 300 may include an electrical impulse generator 310; a power source 320 coupled to the electrical impulse generator 310; a control unit 330 in communication with the electrical impulse generator 310 and coupled to the power source 320; and electrodes 350 coupled to the electrical impulse generator 310 for attachment via leads 340 to one or more selected regions of a mammal. The device 300 may be self-contained or comprised of various separate, interconnected units. The control unit 330 may control the electrical impulse generator 310 for generation of a signal suitable for blocking and/or modulating inhibitory nerve signals when the signal is applied via the electrodes 350 to the nerves. It is noted that SCS device 300 may be referred to by its function as a pulse generator.

By way of example, the drive signals may include at least one of sine waves, square waves, triangle waves, exponential waves, and complex impulses. In one or more embodiments, the signal generator may be implemented using a power source, a processor, a clock, a memory, etc. to produce the aforementioned waveforms, such as a pulse train. The parameters of the drive signal are preferably programmable, such as the frequency, amplitude, duty cycle, pulse width, pulse shape, etc. In the case of an implanted signal generator, programming may take place before or after implantation. For example, an implanted signal generator may have an external device for communication of settings to the generator. An external communication device may modify the signal generator programming to improve treatment.

In the case of contact electrodes, such preferably exhibit impedances permitting a peak pulse voltage in the range from about 0.2 volts to about 20 volts. The blocking and/or modulating signal (current and/or fields) preferably have a frequency, an amplitude, a duty cycle, a pulse width, a pulse shape, etc. selected to influence the therapeutic result, namely blocking some or all of the nerve transmissions in the spinal cord/sympathetic nerve chain.

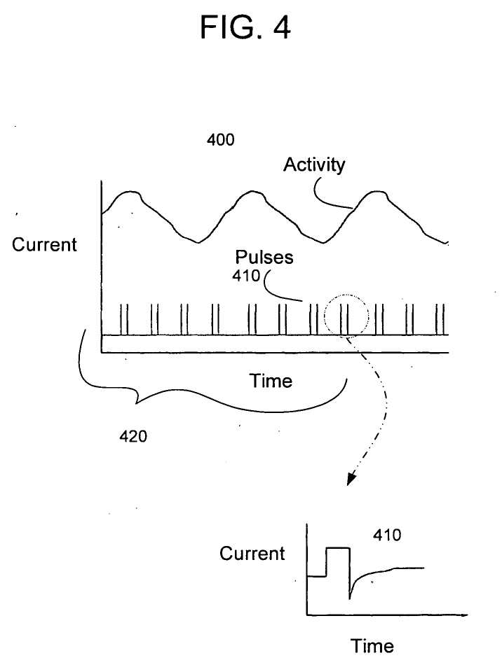

FIG. 4 illustrates an exemplary electrical voltage/current profile for a blocking and/or modulating inhibitory nerve signals in the sympathetic nerve chain. Application of a suitable electrical voltage/current profile 400 may be achieved using the pulse generator 310. In a preferred embodiment, the pulse generator 310 may be implemented using the power source 320 and the control unit 330 having, for instance, a processor, a clock, a memory, etc., to produce a pulse train 420 to the electrode(s) 350 that deliver the blocking impulses 410 to the sympathetic nerve chain via leads 340.

By way of example, the parameters of the drive signal may include a sine wave profile having a frequency of about 10 Hz or greater, such as between about 10-200 Hz, between about 15 Hz to 120 Hz, between about 25 Hz to about 50 Hz, between about 40-65 Hz, and more preferably about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

By way of further example, the parameters of the drive signal may include one or more of a square wave profile having a frequency of about 10 to 60 Hz, more particularly, 10 Hz to 20 Hz, such as 15 Hz. Alternatively, the frequency may be 50 Hz or 25 Hz. The duty cycle of the drive signal may be of less than about 2%, such as between about 0.2 to 1.2%. The peak amplitude of the drive signal may be about 10 to about 20 volts, such as about 10 to about 15 volts. The drive signal may include a pulse width of about 20 us or greater, such as about 100 to about 300 us, for example, 200 us. The electric or electromagnetic field may be administered for a predetermined duration, such as between about 5 minutes and about 1 hour, or between about 5 minutes and about 24 hours.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Among the available devices to implement the control unit and/or signal generator for facilitating the induced current and/or the emission of electric fields and/or electromagnetic fields is a physician programmer, such as a Model 7432 also available from Medtronic, Inc. An alternative control unit, signal generator is disclosed in U.S. Patent Publication No.: 2005/0216062, the entire disclosure of which is incorporated herein by reference. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated. It is noted that if the aforementioned hardware requires modification to achieve the parameters of the drive signals, then one skilled in the art would not require undue experimentation to achieve such modifications, or one skilled in the art would readily be able to obtain hardware capable of producing the drive signals based on the description herein.

### EXPERIMENTAL PROCEDURES

In order to examine the effect of an electric (or electromagnetic) field on the spinal cord in connection with delayed gastrointestinal transit induced by postoperative ileus in a rodent model, experiments were performed on approximately 38 male, Sprague-Dawley rats, weighing between 200-250 g.

Electrode Implantation: Electrodes were implanted in all rats participating in the experiment. The rats were anesthetized with 1.5-3.0% isoflurane, an incision was made to expose the dorsal surface of the vertebral column from T7-T9, additional incisions were made and the skeletal muscle retracted, T8 or T13 were exposed, and a mild laminectomy was performed to expose the dorsal surface of the spinal cord. The cathode of an electrode (2-3 mm in diameter of oblong shape) was positioned between the T7 or T12 process and the spinal dura, and was sutured into place with the skeletal muscle. The anode of an electrode was placed in a subcutaneous pouch along the side of the rat. Finally, the electrode contacts were subcutaneously placed at the base of the neck and exposed via a small incision. The rats were then returned to home cages and allowed to recover for 3-5 days.

Post-Operative Ileus: Following an overnight fast (of between 18-20 hrs) a laparotomy was performed in all rats. The rats were anesthetized via isoflurane (5-2%) inhalation and the abdominal region was shaved and prepared for surgery, and the rats were placed on a heating blanket to maintain proper body temperature, and a mid-line incision (2.5-3.5 cm) was made. After laparotomy the intestine and the cecum were exteriorized on saline-soaked gauze and the intestines were gently manipulated (inspected with two cotton applicators that were soaked in saline) to resemble the running of the bowel performed in patients during abdominal surgery. The organs were then placed back into the abdominal cavity and the muscle and skin layers were closed with silk suture.

Control Groups: In order to establish scientific controls during experimentation, one control group (GROUP I) did not have post-operative ileus surgery, had electrodes implanted, and received no electric or electro-magnetic fields to the spinal cord during experiments. Another control group (GROUP II) had post-operative ileus surgery, had electrodes implanted, but received no electric or electro-magnetic fields to the spinal cord during experiments.

Measurements during experimentation were taken on gastric emptying, and small intestinal transmit, expressed as a geometric center of the meal and the head of the meal.

Gastric emptying: Gastric emptying was measured according to the method disclosed in Trudel et al., AJP-Gastrointest Liver Physiol 282:G948 (2002). In particular a 1.5% methylcellulose solution (in distilled H₂O) and ^{99m}Tc (approximately 100,000 cpm) was administered intragastrically through a stainless steel gavage needle. After receiving the radioactive meal, the rats were placed in a wire-bottom cage without access to food and water to allow transit of the intragastric content. All rats were euthanized by CO₂ inhalation 15 minutes after receiving the meal. The abdomen was then opened via a midline incision; and the stomach secured with a single silk ligature at the esophageal junction and two parallel ligatures between the pyloric junction and the duodenum. The stomach with the entire length of the small intestine attached was isolated and arranged on a flat surface and graphed from 0 to 140 cm. The total length of the small intestine was measured. Intestinal segments (of 10 cm each) were separated by ligatures starting from the pyloric junction. The stomach and the intestinal sections were numbered (1, 2, 3 etc., and excised starting with the duodenum) and placed in separate test tubes. Radioactivity remaining in the stomach and in the intestinal segments, arranged in proximal to distal order, were measured using a gamma-counter. As will be discussed below, the data (c.p.m.) are expressed as a percentage of the total recovered radioactivity. The gastric emptying was measured by the percentage of total recovered radioactivity remaining in the stomach 15 minutes after the intragastric infusion.

Small intestinal transit: The small intestinal transit was assessed by the geometric center calculated as a function of the amount of intragastric content transported to each segment along the intestine. Calculations were made according to the method of Miller developed in 1981:

GC=Σ(% radioactivity per segment×number of segment)/100.

The small intestinal transit was also characterized by the maximal distance (cm) reached by the head of the meal along the length of the small intestine.

Spinal cord signal modulation: For those rats that received stimulation, the parameters used for such modulation were administered by driving the cathode and anode electrodes with a square wave signal of 200 us (on-time) and an off time established by the signal frequency, which was adjusted to 15 Hz, 25 Hz and 50 Hz during different experiments. The duration of the signal application was 15 minutes. The amplitude of the signal applied to the electrodes was determined by increasing the voltage/current until the onset of motor threshold (muscle twitching) in a given rat, and then the voltage/current was reduced to 90% of such level.

For upper gastrointestinal transit, spinal cord signal modulation started immediately following administration of the radioactive meal and continued until euthanasia (15 minutes later).

For colonic transit, stimulation may start after the minute recovery from the injection of the dye and may be applied in cycles—60 minutes of stimulation followed by 60 minutes of non-stimulation until the appearance of fecal pellets with the dye.

With respect to a first set of experiments, the results of the experiments for gastric emptying, and small intestinal transmit (both geometric center and head of meal), GROUP I rats (i.e., those rats that did not have post-operative ileus surgery) provided a baseline indicating a relatively normal digestive function—expressing no postoperative ileus. GROUP II rats (i.e., those rats that had post-operative ileus surgery, but no spinal cord modulation) exhibited digestive abnormalities expressed as significant postoperative ileus.

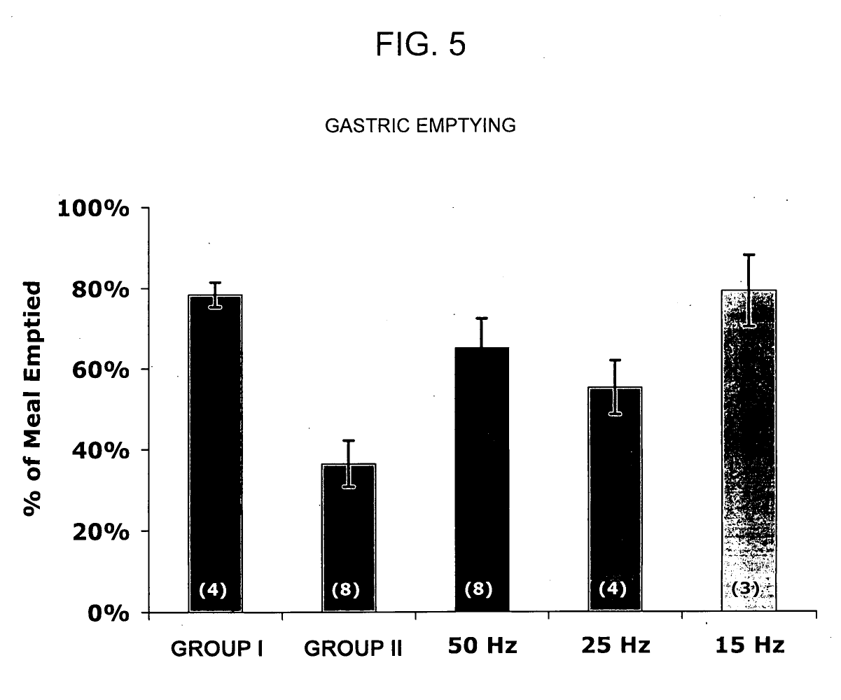

Turning to FIG. 5, the results of determining the emptying of the stomach of the experimental rats show that spinal cord modulation over the range of about 15 Hz to about 50 Hz resulted in significant improvement of the digestive function expressed as gastric emptying. Indeed, the rats treated with 15 Hz (80% empty) signaling exhibited gastric emptying at approximately the same level as the GROUP I rats. The rats treated with 50 Hz (65% empty) signaling exhibited the next best result, while the rats treated with 25 Hz (55% empty) signaling followed (still a 20% improvement).

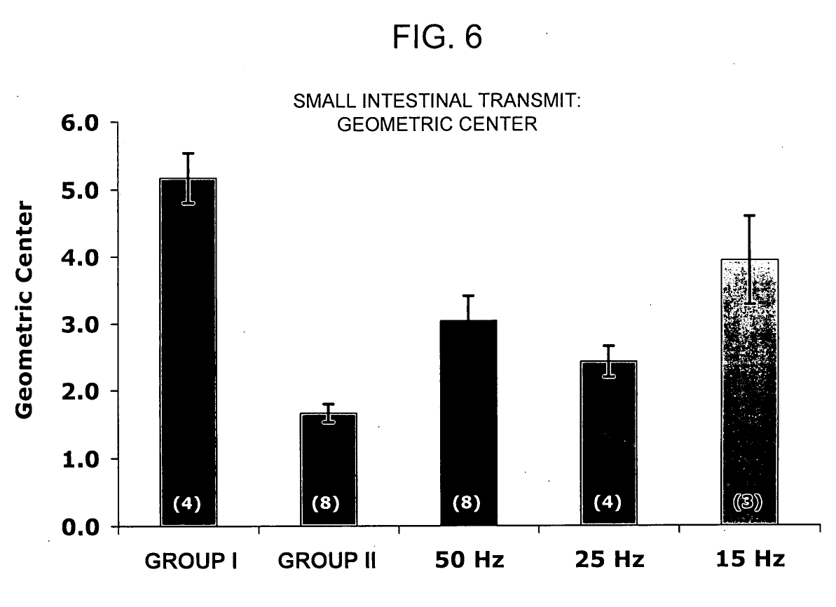

Turning to FIG. 6, the results of determining the geometric center of the meal also show that spinal cord modulation over the range of about 15 Hz to about 50 Hz resulted in significant improvement of the digestive function. Indeed, the rats treated with 15 Hz signaling exhibited a geometric center only about 1-1.5 units behind the GROUP I rats. The rats treated with 50 Hz signaling exhibited the next best result, while the rats treated with 25 Hz signaling followed. Again, all treatment frequencies improved the digestive function.

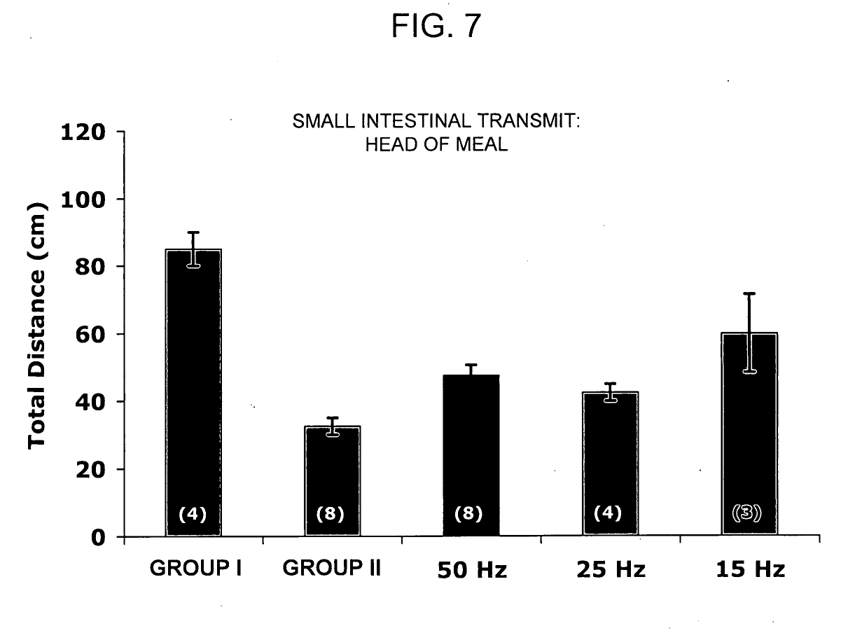

Finally, turning to FIG. 7, the results of determining the head of meal also show that spinal cord modulation over the range of about 15 Hz to about 50 Hz resulted in improvement of the digestive function. The rats treated with 15 Hz signaling exhibited a head of meal only about 25 cm behind the GROUP I rats. The rats treated with 50 Hz signaling exhibited the next best result, while the rats treated with 25 Hz signaling followed.

With respect to a second set of experiments, male Sprague Dawley rats weighing 260-510 g, housed under controlled conditions (21° C., 0600-1800 light/dark cycle) with ad libitum availability to water and standard rat chow were used. Animals were fasted for 18-24 hours with free access to water prior to all gastric emptying/upper GI transit experiments; aseptic technique was employed in all surgical procedures.

For two surgical manipulations, rats were anesthetized with 5% Isoflurane and the surgical site was shaved and prepared with three alternating scrubs of betadine and 70% isopropyl alcohol. The surgical site for electrode implantation was the dorsal region from the lower thoracic spine to the base of the skull. The surgical site for POI was the ventral surface of the abdomen. Body temperatures were maintained throughout each surgical procedure at 37° C. using a homeothermic heating blanket. Heart rate and pO₂ were monitored with a SurgiVet Advisor monitor (Smiths Medical, Waukesha, Wis.) and adequate anesthesia was maintained with 2% Isoflurane.

Following surgical preparation, a 2.5 cm sagittal incision was made dorsal to the T₂-T₅ spinous processes and the paravertebral musculature was retracted laterally. Once the upper thoracic spinous processes were exposed, a laminectomy was performed at T₄ to expose the spinal dura. A rectangular (2×3 mm) cathode was gently teased into the epidural space of the neural canal between the T₅ and T₈ segmental levels. The paravertebral musculature was returned to its original position and the cathode lead wire secured in place by suturing both sides of the paravertebral muscles together with 3-0 nylon. A subcutaneous pouch was formed from the sagittal incision to the left lateral thoracic cage by teasing the overlying skin from the underlying subcutaneous musculature in the myofascial plane and the circular anode (diameter=5 mm) placed deep within this pocket. A 1.0 cm transverse incision was placed at the level of the upper cervical spine and a subcutaneous tunnel was teased between the overlying skin and the underlying subcutaneous musculature in the myofascial plane caudally to the level of the initial incision. The contact for the electrodes was pulled rostrally through this tunnel and secured in place by closing the transverse incision around the contact with 3-0 nylon thereby exposing the contacts for external manipulation. The sagittal incision was then closed with 3-0 nylon, triple antibiotic ointment applied liberally to the incision sites, and rats were returned to individual cages with free access to standard rat chow and a solution of 2 mg/ml acetaminophen and water for 4-7 days for post-surgical recovery.

To experimentally induce POI, a laparotomy and cotton applicator manipulation of the small intestines, cecum and proximal colon were employed to simulate the ‘running of the bowel’ as performed in surgical procedures of the abdomen, as described previously (Venkova et al., 2007; adapted from Kalff et al, 1998). Following surgical preparation, a 3 cm sagittal incision opening of the abdomen was made and the small intestine, cecum, and proximal aspect of the colon were exteriorized onto sterile gauze that had been moistened with warm (37° C.) sterile normal saline solution. The intestines were then gently manipulated along the exteriorized length for 5 minutes with two cotton swabs which had been soaked in warm (37° C.) sterile normal saline; the exteriorized intestines were covered with gauze and moisture maintained with warm (37° C.) sterile normal saline solution for a period of 5 minutes. The exteriorized intestines were then returned to the abdominal cavity, the abdominal musculature was closed with 4 to 6 sutures of 3-0 nylon, and the abdominal skin closed with a running stitch of 6 loops with 3-0 nylon. The POI rats were then returned to their cages, the appropriate (+/−) SCS leads were connected to the external contact, and the rats were allowed to fully regain consciousness.

The spinal cord stimulation system used in these experiments has proven dependable in many previous animal studies. The stimulation current was generated by a Grass stimulator (S88X) connected to a constant voltage stimulus isolation unit (SIU-V) connected to a constant current unit (CCU 1) (AstroMed Inc, West Warwick, R.I.). The stimulation parameter used for SCS was similar to that used clinically in man and consisted of monophasic rectangular pulses (15 to 200 Hz; pulse width 0.2 ms) with intensity set at 90% of motor threshold (tonic contraction of the abdominal muscles).

The experimentally induced POI rats were allowed to be fully conscious for a period of 15 minutes prior to commencing SCS testing. During the recovery phase, the individualized motor threshold was determined by applying an increasing test current at 7 Hz until involuntary skeletal muscle twitch at the stimulation rate was observed. The test stimulation was then stopped, the current required to reach motor threshold was measured and adjusted to 90% of the required value. The stimulator was then set to deliver pulses at one of the experimental frequencies (15-200 Hz) for SCS.

Upon completion of the 15 min recovery phase, the rats received a meal via intragastric gavage of 1.5 ml ^{99m}Tc (adjusted to approximately 100,000 counts per minute (c.p.m.) in 1.5% methylcellulose/distilled water solution. Rats, except sham or naïve groups, then received SCS at the specified parameters. Fifteen minutes after the start of the stimulation, the rats were euthanized by CO₂ inhalation, and the abdomen reopened via a sagittal incision.

The gastroesophageal junction was located and ligated with a single silk ligature, the pyloroduodenal junction was ligated with two parallel silk ligatures, and the ileocecal junction was ligated with a single silk ligature. The esophagus was cut superior to the gastroesophageal ligature and a cut was placed immediately distal to the ligature at the ileocecal junction. The stomach and small intestine were carefully removed from the abdominal cavity, laid out on paper that had been graphed from 0 to 140 cm, measured, and the total length of the small intestine was recorded. The stomach was separated from the small intestine by cutting between the parallel ligatures mentioned above and then sectioned into three smaller segments. Each segment was placed in a labeled test tube; the scissors used to cut the stomach and the weighing dish in which it was sectioned were carefully rinsed with distilled water which was then added to the test tubes containing the gastric sample. Commencing from the pyloroduodenal junction, ligatures were tied every 10 cm thereby dividing the intestine into equal length segments. The ligated small intestine was clamped off immediately superior to a ligature and cut between the clamp and the ligature to ensure no leakage of the intestinal contents. The segments were then placed into labeled test tubes for counting. Radioactivity remaining in the stomach and in the intestinal segments, arranged in proximal to distal order, was measured using a gamma-counter. The data (c.p.m.) was expressed as percentage of the total recovered radioactivity. Gastric emptying was measured as the percentage of total recovered radioactivity remaining in the stomach 15 minutes after the intragastric infusion and stimulation. The small intestinal transit was assessed by the geometric center calculated as a function of the amount of intragastric content transported to each segment along the intestine. Calculations were made according to the method of Miller and coworkers (1981): geometric center=Σ(% of total radioactivity per segment×number of segment)/100. The small intestinal transit was characterized also by the maximal distance (cm) reached by the head of the meal along the length of the small intestine, measured as the most distal 10 cm segment with activity greater than 3× background activity.

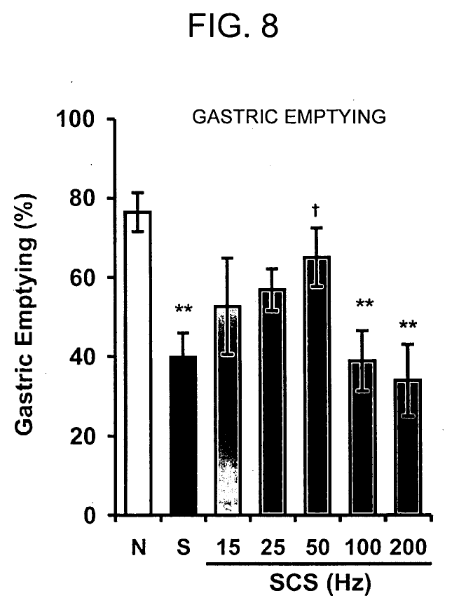

With reference to FIGS. 8-10, the data are expressed as the mean±standard error of the mean (S.E.M.). Statistical significance, assigned if p<0.05, was assessed between different groups using a one-way analysis of variance (ANOVA) with a Bonferroni's post-test to determine differences between groups.

In freely moving control rats with the spinal electrodes implanted but not electrically stimulated (sham-stimulated) it has been demonstrated that surgical manipulation of the bowel was associated with a significant reduction in gastric emptying of a radioactive meal compared to naïve rats that had not undergone surgery to induce POI. FIG. 8 shows the gastric emptying rates, expressed as a percentage of the radiolabeled meal emptied from the stomach. In the sham-stimulated group (S), POI surgery delayed gastric emptying as demonstrated by significantly less of the radiolabeled meal exiting the stomach compared to the non-treated naïve control group (N).

Having demonstrated a delay in gastric emptying induced by surgical manipulation of the bowel, examination was made as to whether SCS had any affect on delayed gastric emptying. In separate experiments it was found that SCS (15-50 Hz) reversed the delay in gastric emptying to levels that were not statistically different from the naïve control group (FIG. 8). Additionally at 50 Hz the percentage of the meal emptied from the stomach differed significantly from the sham control group with POI in which the SCS electrodes had been implanted but not stimulated.

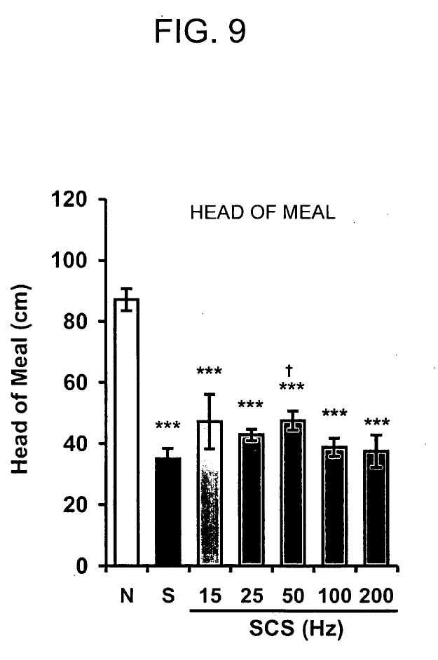

Having shown these significant effects of SCS to reverse the delay in gastric emptying induced by surgical manipulation of the bowel, examination as to the effect of induced-POI on small intestinal transit as measured as the head of the radioactive meal was conducted. As illustrated in FIG. 9, the sham stimulation control group (S) with POI had significantly slower small intestinal transit compared to the naïve group (N), whereas the SCS group demonstrated significantly improved small intestinal transit compared with the sham group, but did not achieve a normalization of transit when compared to the naïve group. An additional measure of transit of the meal, the geometric center of the meal, i.e., the location at which most of the meal is concentrated was significantly reduced in the sham stimulation control group with POI compared to the naïve group.

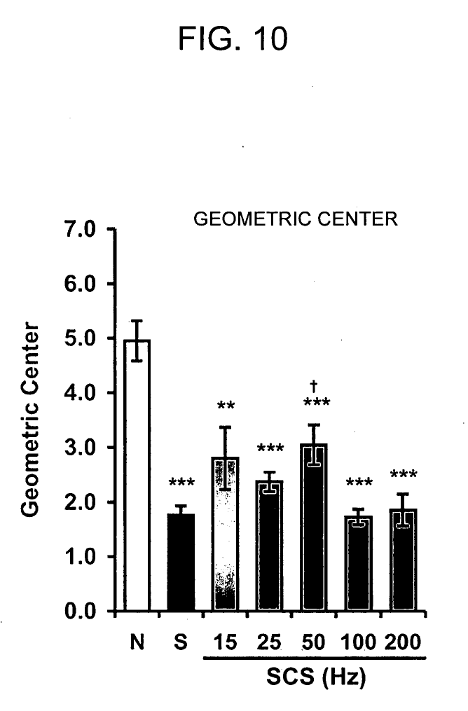

As shown in FIG. 10, while 50 Hz SCS significantly increased the geometric center compared to the sham group, the overall effect of SCS was still significantly less than the naïve group.

The results of the present study reveal that SCS had a positive effect on delayed gastric emptying produced by surgical manipulation of the bowel in a rodent model. The observed normalization of gastric emptying occurred at the 15, 25 and 50 Hz setting of electrical stimulation with the greatest degree of normalization occurring at 50 Hz. The data generated in this study also demonstrated that the geometric center of the radiolabeled meal was also improved with 50 Hz SCS. While the observed data is not quite as robust as that noted in gastric emptying, the finding, however, remains compelling for the effect of SCS. The data collected for the head of the meal, in the present study, was the least robust of the three parameters observed with upper GI transit. Despite the fact that 50 Hz had a modest improvement with respect to the head of the meal, it must be remembered that SCS was only applied for 15 minutes and a longer period of stimulation may improve the head of meal finding.

In the current study a systematic investigation of SCS on gastric and intestinal motility was performed using multiple frequencies of electrical stimulation ranging from 15-200 Hz. It has been shown that the most effective frequency of SCS was 50 Hz, which is in the same range (40-65 Hz) as that employed to modulate gastrointestinal motility in the clinical reports (18; 20). Furthermore, this frequency of SCS is identical to that used previously to treat pain of gastrointestinal origin induced by colorectal distension in a rodent model. Taken together, the results obtained in this study provide important evidence of a possible non-pharmacological therapeutic approach to the treatment of delayed gastric and/or small intestine motor function producing postoperative gastric ileus secondary to abdominal surgery. Without limiting the invention to any particular theory of operation, the underlying mechanisms responsible for the stimulatory effects of SCS in the current experimental model suggest synaptic modification in spinal and supraspinal pathways. In support of a central mechanism SCS suppresses pathological hyperexcitability of wide dynamic range spinal neurons after peripheral nerve lesions. Furthermore, evidence in a rodent model of peripheral vasodilation of the prior art suggests that SCS depresses sympathetic nervous activity, which may account for the reversal in delay in upper GI transit observed in the current study. SCS may thus play a role in shifting the autonomic neural regulation of GI motility by attenuating inhibitory sympathetic tone that is enhanced in response to abdominal manipulation. In fact chemical sympathetic blockage by perioperative epidural analgesia in patients and intraperitoneal administration of guanethidine in mice has been shown to promote gastric motility and reverse POI. These studies along with the two case report studies cited earlier where SCS was associated with the relief from constipation and the adverse diarrhea symptoms, suggests that the increased GI motility following SCS may be associated with a release from sympathetic inhibition.

Recent studies in the art have shown that SCS also acts to increase blood flow via antidromic activation of sensory afferents to release neuromodulatory substances at the target organ. Such a mechanism may also play a role in the effect of SCS in the current experimental models. Furthermore, SCS may possibly inhibit neurons within the intrinsic ganglia of the enteric nervous system that become sensitized in response to POI. In support of this hypothesis, recent observations in the art have shown that SCS applied at the T₁-T₂ level depresses the activity generated by intrinsic cardiac neurons, which was most evident after provocation of the cardiac neurons with local ischemia.

POI is a poorly treated yet common problem that contributes to patient morbidity and results in prolonged hospital stays following surgery. Taken together, the results of the current study demonstrate SCS normalizes gastric emptying and improves upper GI transit in a rodent model of POI.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a temporary arrest of intestinal peristalsis, comprising inducing at least one of an electric current, an electric field and an electromagnetic field in a sympathetic nerve chain of a mammal to block and/or modulate inhibitory nerve signals thereof such that intestinal peristalsis function is at least partially improved.

2. The method of claim 1, wherein the electric current, electric field and/or electromagnetic field is applied to at least one of the celiac ganglia, cervical ganglia, and thoracic ganglia of the sympathetic nerve chain.

3. The method of claim 1, wherein the electric current, electric field and/or electromagnetic field is applied to at least a portion of the splanchnic nerves of the sympathetic nerve chain.

4. The method of claim 1, wherein the electric current, electric field and/or electromagnetic field is applied to a spine of the mammal at one or more of the levels from T5 to L2.

5. The method of claim 1, further comprising inducing the current and/or field(s) by applying at least one electrical impulse to one or more emitters.

6. The method of claim 5, wherein the one or more emitters are disposed at least one of subcutaneously and percutaneously to direct the field(s) to the sympathetic nerve chain.

7. The method of claim 6, wherein the one or more emitters include at least one of contact electrodes, capacitive coupling electrodes, and inductive coils.

8. The method of claim 5, further comprising applying drive signals to the one or more emitters to produce the at least one impulse and induce the current and/or field(s).

9. The method of claim 8, wherein the drive signals include at least one of sine waves, square waves, triangle waves, exponential waves, and complex impulses.

10. The method of claim 8, wherein the drive signals are square waves.

11. The method of claim 9, wherein the drive signals include a frequency of about 10 to 200 Hz.

12. The method of claim 11, wherein the frequency is between about 10 Hz to 20 Hz.

13. The method of claim 11, wherein the frequency is about 15 Hz.

14. The method of claim 11, wherein the frequency is about 40-65 Hz.

15. The method of claim 11, wherein the frequency is about 50 Hz.

17. The method of claim 9, wherein the drive signals include a duty cycle of between about 0.2 to 1.2%.

18. The method of claim 9, wherein the drive signals include a peak amplitude of about 10 to about 20 volts.

19. The method of claim 18, wherein the peak amplitude is between about 10 to about 15 volts.

20. The method of claim 9, wherein the drive signals include a pulse width of about 20 us or greater.

21. The method of claim 18, wherein the pulse width is between about 100 to about 300 us.

22. The method of claim 21, wherein the pulse width is about 200 us.

23. The method of claim 1, further comprising inducing the electric or electromagnetic field for a predetermined duration.

24. The method of claim 23, wherein the predetermined duration is between about 5 minutes and about 1 hour.

25. The method of claim 23, wherein the predetermined duration is between about 5 minutes and about 24 hours.
